(19) United States
(12) Patent Application Publication
(10) Pub. No.: US 2015/0037852 A1
(43) Pub. Date: Feb. 5, 2015

(54) METHOD AND SYSTEM FOR REDUCING FREE FATTY ACID CONTENT OF A FEEDSTOCK

(71) Applicants: Lu-Kwang Ju, Akron, OH (US); Majid Hosseini, Cuyahoga Falls, OH (US)

(72) Inventors: Lu-Kwang Ju, Akron, OH (US); Majid Hosseini, Cuyahoga Falls, OH (US)

(21) Appl. No.: 14/450,601

(22) Filed: Aug. 4, 2014

Related U.S. Application Data
(60) Provisional application No. 61/861,507, filed on Aug. 2, 2013.

(51) Int. Cl. C11B 3/02; C11B 13/00; C11C 3/00; C12P 7/64

(52) U.S. Cl. CPC C11B 3/02; C12P 7/649; C11B 13/00; C11C 3/00
USPC 435/134; 435/280; 554/167; 554/175; 554/163

(57) ABSTRACT

A method for reducing the free fatty acid content of a feedstock includes the steps of providing a free-fatty-acid-containing feedstock, treating the free-fatty-acid-containing feedstock to reduce the free fatty acid content thereof, where the step of treating includes combining at least one of an algae and a coagulant to the free-fatty-acid-containing feedstock, and producing a product from the treated feedstock.

### CROSS-REFERENCE TO RELATED APPLICATION

This application claims priority to U.S. Provisional Patent Application No. 61/861,507 filed on Aug. 2, 2013, the contents of which are incorporated herein by reference.

### FIELD OF THE INVENTION

The present invention generally relates to reducing the free fatty acid (FFA) content of a feedstock. The present invention further relates to reducing the free fatty acid content of a biodiesel feedstock by treating the feedstock with algae. The present invention further relates to reducing the free fatty acid content of a biodiesel feedstock by treating the feedstock with a coagulant to form a coagulant complex with the free fatty acid.

### BACKGROUND OF THE INVENTION

Biodiesel is one alternative source of energy to petroleum-based fuels that can help overcome some of the current energy problems, such as rising energy prices, increasing energy demand, and changing climate due to CO₂ accumulation from burning fossil fuels. However, the cost of biodiesel remains relatively high and raw material costs can make up approximately 70%-85% of the total biodiesel production cost. Thus, using cheaper feedstock is very helpful for making the production of biodiesel more economical.

Waste cooking oil (WCO) is cheap and widely available as an economic biodiesel feedstock. There are also other potential oils and lipid sources that are available as cheaper biodiesel feedstock. However, these cheaper feedstocks often have higher free fatty acid (FFA) content and cannot be directly transesterified to biodiesel by the more effective and economic alkali-catalyzed process. This is because the presence of water and FFA creates an emulsion or a soap during transesterification and negatively affects the catalyst efficiency, production rate, and yield.

Pretreatment steps for FFA-containing feedstocks are known. Such steps include esterification by acid catalysts or enzymatic catalysts, co-solvent methods, steam distillation, alcohol extraction, and a glycerolysis reaction. However, such pretreatments are generally costly and are not feasible for industrial scale processes.

Another known pretreatment process includes neutralization or caustic pretreatment of the FFA. However, this pretreatment results in a large loss of neutral oil as a result of the emulsification step that is involved. Also, other disadvantages of this method include the need for a mechanical force, such as centrifugation, to remove the emulsified oil and the need to treat the resulting oily component.

Thus, there is a need in the art for an improved method and system for lowering the FFA content of biodiesel feedstocks. There is a further need for an improved method and system for pretreating biodiesel feedstocks that are economically attractive and industrially viable to allow biodiesel production from FFA-containing feedstocks, particularly high FFA feedstocks.

### SUMMARY OF THE INVENTION

In a first embodiment, the present invention provides a method for reducing the free fatty acid content of a feedstock comprising the steps of combining algae with a medium to form an algae-containing medium, combining a free-fatty-acid-containing feedstock with the algae-containing medium to form an algae treatment feedstock, allowing the algae to reduce the free-fatty-acid content of the algae treatment feedstock, and, after said step of allowing, producing a product from the algae treatment feedstock.

In a second embodiment, the present invention provides a method as in the first embodiment, wherein the medium comprises water, the method further comprising the steps of agitating the algae treatment feedstock to form an upper oil phase, a lower aqueous phase, and a middle phase and collecting said upper oil phase.

In a third embodiment, the present invention provides a method as in either the first or second embodiment, wherein the collected upper oil phase has a lower free fatty acid content than the free-fatty-acid-containing feedstock.

In a fourth embodiment, the present invention provides a method as in any of the first through third embodiments, wherein the collected upper oil phase has a lower acid value than the free-fatty-acid-containing feedstock.

In a fifth embodiment, the present invention provides a method as in any of the first through fourth embodiments, wherein the medium comprises water and waste cooking oil.

In a sixth embodiment, the present invention provides a method as in any of the first through fifth embodiments, wherein the medium comprises a mineral medium.

In a seventh embodiment, the present invention provides a method as in any of the first through sixth embodiments, wherein the mineral medium comprises one or more of the following components: glucose, MgCO₃, NH₄Cl, KH₂PO₄, nitrilotriacetic acid, peptone, MgSO₄.7H₂O, yeast extract, CaCO₃, Na₂EDTA, FeCl₃.6H₂O, H₃BO₃, thiamine, MnCl₂.4H₂O, ZnSO₄.7H₂O, CoCl₂.6H₂O, Na₂MoO₄.2H₂O and biotin.

In an eighth embodiment, the present invention provides a method as in any of the first through seventh embodiments, wherein the mineral medium comprises 10 g/L glucose, 0.4 g/L MgCO₃, 0.3 g/L NH₄Cl, 0.3 g/L KH₂PO₄, 0.2 g/L nitrilotriacetic acid, 0.5 g/L peptone, 0.1 g/L MgSO₄.7H₂O, 0.5 g/L yeast extract, 0.05 g/L CaCO₃, 4.4 mg/L Na₂EDTA, 3.15 mg/L FeCl₃.6H₂O, 0.97 mg/L H₃BO₃, 0.25 mg/L thiamine, 0.18 mg/L MnCl₂.4H₂O, 0.02 mg/L ZnSO₄.7H₂O, 0.01 mg/L CoCl₂.6H₂O, 6 μg/L Na₂MoO₄.2H₂O and 2.5 μg/L biotin.

In a ninth embodiment, the present invention provides a method as in any of the first through eighth embodiments, further comprising one or more of the steps consisting of autoclaving the mineral medium, filtering particles from the free-fatty-acid-containing feedstock, and aerating the algae treatment feedstock.

In a tenth embodiment, the present invention provides a method as in any of the first through ninth embodiments, further comprising the step of providing a surfactant to the medium.

In an eleventh embodiment, the present invention provides a method as in any of the first through tenth embodiments, wherein the produced product is biodiesel.

In a twelfth embodiment, the present invention provides a method for reducing the free fatty acid content of a feedstock comprising the steps of combining a coagulant with a free-fatty-acid-containing feedstock and forming a coagulant complex including the coagulant and free fatty acid.

In a thirteenth embodiment, the present invention provides a method as in any of the first through twelfth embodiments, wherein the free-fatty-acid-containing feedstock has a pH of 5 or greater.

In a fourteenth embodiment, the present invention provides a method as in any of the first through thirteenth embodiments, further comprising the step of adding a base to the free-fatty-acid-containing feedstock to bring the feedstock to a pH of from 5 to 10.

In a fifteenth embodiment, the present invention provides a method as in any of the twelfth through fourteenth embodiments, further comprising the steps of forming phase separations including an upper oil phase, a middle phase, and a lower aqueous phase, where the middle phase includes the coagulant complex, collecting the upper oil phase, and using the upper oil phase to produce a product.

In a sixteenth embodiment, the present invention provides a method as in any of the first through fifteenth embodiments, wherein the produced product is biodiesel.

In a seventeenth embodiment, the present invention provides a method as in any of the first through sixteenth embodiments, wherein a method for producing biodiesel comprising the steps of providing a free-fatty-acid-containing feedstock, treating the free-fatty-acid-containing feedstock to reduce the free fatty acid content thereof, where the step of treating includes combining at least one of an algae and a coagulant to the free-fatty-acid-containing feedstock, forming phase separations in the treated feedstock, the phase separations including an upper oil phase, a middle phase, and a lower aqueous phase, collecting the upper oil phase, and producing a biodiesel product from the upper oil phase.

In an eighteenth embodiment, the present invention provides a method as in any of the first through seventeenth embodiments, wherein said step of producing the biodiesel product includes extracting lipids and reacting the lipids with an alcohol using a chemical or enzymatic process.

In a nineteenth embodiment, the present invention provides a method as in any of the first through eighteenth embodiments, wherein the alcohol is selected from the group consisting of methanol and ethanol.

### DETAILED DESCRIPTION OF ILLUSTRATIVE EMBODIMENTS

The present invention generally relates to reducing the free fatty acid (FFA) content of a composition. The present invention further relates to reducing the free fatty acid content of a biodiesel feedstock by treating the feedstock with algae. The present invention further relates to reducing the free fatty acid content of a biodiesel feedstock by treating the feedstock with a coagulant to form a coagulant complex with the free fatty acid. As used herein, feedstock means any raw material or input stream of a process. As used herein, FFA-containing feedstock means any raw material or input stream of a process where the raw material or input stream contains free fatty acid. In one or more embodiments, a feedstock is fed into a process for treatment or for conversion into something different. In one or more embodiments, a feedstock is used to produce biodiesel.

In the present invention, a medium comprises at least one free fatty acid. The medium is also described herein as FFA-containing feedstock. In embodiments where algae are utilized, the FFA-containing feedstock must comprise water as algae need water to survive. An FFA-containing feedstock may comprise other components in addition to the at least one free fatty acid. In embodiments where algae are utilized and where it is desired for the algae to grow or reproduce, an FFA-containing feedstock can provide nutrients essential for the algae to grow or reproduce. In one or more embodiments, essential nutrients can be added to the FFA-containing feedstock. These essential nutrients are those that allow the algae to grow or reproduce and these nutrients are not required in embodiments where it is not desired for the algae to grow or reproduce. In embodiments where algae are utilized and where it is not desired for the algae to grow or reproduce, the FFA-containing feedstock is only required to include at least one free fatty acid and water.

In embodiments where essential nutrients are added, a mineral medium comprises these essential nutrients. In these embodiments, a mineral medium is combined with the FFA-containing feedstock. In one or more embodiments, a mineral medium comprises one or more of the following components: glucose, MgCO₃, NH₄Cl, KH₂PO₄, nitrilotriacetic acid, peptone, MgSO₄.7H₂O, yeast extract, CaCO₃, Na₂EDTA, FeCl₃.6H₂O,

H₃B0₃, thiamine, MnCl₂.4H₂O, ZnSO₄.7H₂O, CoCl₂.6H₂O, Na₂MoO₄.2H₂O, and biotin. In one or more embodiments, the mineral medium includes 10 g/L glucose, 0.4 g/L MgCO₃, 0.3 g/L NH₄Cl, 0.3 g/L KH₂PO₄, 0.2 g/L nitrilotriacetic acid, 0.5 g/L peptone, 0.1 g/L MgSO₄.7H₂O, 0.5 g/L yeast extract, 0.05 g/L CaCO₃, 4.4 mg/L Na₂EDTA, 3.15 mg/L FeCl₃.6H₂O, 0.97 mg/L H₃BO₃, 0.25 mg/L thiamine, 0.18 mg/L MnCl₂.4H₂O, 0.02 mg/L ZnSO₄.7H₂O, 0.01 mg/L CoCl₂.6H₂O, 6 μg/L Na₂MoO₄.2H₂O, and 2.5 μg/L biotin. In one or more embodiments, a mineral medium can comprise one or more of the above amounts in an approximation to the amounts given above.

One or more embodiments of the present invention provide a method and system for treating a carbon source with algae for reducing the FFA content of the carbon source. One or more embodiments of the present invention provide a method and system for reducing the FFA content of a carbon source where the carbon source is a high FFA oil. In these embodiments, a FFA-containing oil, such as waste cooking oil (WCO), is combined with microalgae in a mineral medium. This composition can be referred to as an algae treatment composition. While it is envisioned that phagotrophic algae are preferred for the present invention, any suitable algae that preferentially reduce the FFA from an oil mixture of FFA, triglycerides, and lipids, can be utilized.

It is envisioned that where algae is used in the present invention, the algae are preferably phagotrophic algae. The phagotrophic algae can also perform other functions and can therefore also have other classifications in addition to phagotrophic. Such classifications can include photosynthetic, heterotrophic, and osmotrophic. The algae may have more than one of these classifications.

Phagotrophic algae are those that grow by engulfing their food, photosynthetic algae are those that grow by using light as the energy source, heterotrophic algae are those that feed on organic substrates, and osmotrophic algae uptake dissolved compounds through a membrane via osmosis or other active transport mechanisms (excluding phagotrophy) across the membrane.

Phagotrophic algae are algae that feed by engulfing their food, similar to the function of a mouth. Without being limited to a particular theory, it is envisioned that the phagotrophic algae preferentially reduce the FFA from an oil mixture of FFA, triglycerides, and lipids. Phagotrophic algae can engulf oil particles and preferentially digest the FFA with special enzymes that they keep inside their bodies without sharing the food generated with others.

Where phagotrophic algae are utilized, oil ingested can be seen under microscope as individual droplets inside the cells, without extensive micellar solubilization or pre-catabolic hydrolysis. Without being limited to a particular theory, it is hypothesized that the alga preferentially consumes the more water-soluble components, including fatty acids, in the oil feedstock (e.g. WCO) over the more hydrophobic glycerides. Therefore, controlled growth of the alga combined with the oil feedstock (e.g. WCO) leads to reduced FFA content in the remaining oil.

In one or more embodiments, the algae are preferably of the Ochromonas danica microalgae species. Microalga Ochromonas danica are commonly in a tear-drop or spherical shape and has two unequal flagellates and one chloroplast. The alga can grow on soluble organics (heterotrophy) and by photosynthesis. It can also grow phagotrophically by engulfing oil droplets, particles, and bacteria. This alga can also grow on substrates such as starch grains, casein, small organisms, phenols, phenolic mixtures, synthetic lubricants, fats, alcohols, carboxylic acids and amino acids.

Other suitable phagotrophic algae may be chosen from several chrysomonad genera including Dinobryon, Chrysophaerella, Uroglena, Catenochrysis, Ochromonas, Chromulina, and Chrysococcus; the prymnesiophyte Chrysochromulina; the coccolithophorid Coccolithus pelagicus; the xanthophyte Chlorochromonas, the chrysophytes Phaeaster, Chrysamoeba, and Pedinella; the dinoflagellate Ceratium hirundinella Muller; and Cryptomonas ovata Ehrenberg.

In one or more embodiments, the algae are selected from Chlorella and Ochromonas species. In one or more embodiments, the algae are Ochromonas species. In one or more embodiments, the algae are Chlorella species.

Other suitable phagotrophic may be chosen from Dinobryon chrysomonads, Chrysophaerella chrysomonads, Uroglena chrysomonads, Catenochrysis chrysomonads, Ochromonas chrysomonads, Chromulina chrysomonads, Chrysococcus chrysomonads, Chrysochromulina prymnesiophytes, Coccolithus pelagicus coccolithophorids, Chlorochromonas xanthophytes, Phaeaster chrysophytes, Chrysamoeba chrysophytes, Pedinella chrysophytes, Ceratium hirundinella, and Cryptomonas ovate.

Still other suitable phagotrophic may be chosen from Ochromonas species including Ochromonas malhamensis, Ochromonas tuberculata, Ochromonas vallescia, and other Ochromonas chrysophytes.

As discussed above, the algae are preferably phagotrophic, but can also be described by other classifications. Photosynthetic are those algae that utilize light as their energy source through the process of photosynthesis. Heterotrophic algae are those that feed on organic substrates.

Osmotrophic algae uptake dissolved compounds through a membrane via osmosis or other active transport mechanisms (excluding phagotrophy) across the membrane. Suitable osmotrophic algae may be chosen from Achnanthes orientalis, Agmenellum, Amphiprora hyaline, Amphora coffeiformis, Amphora coffeiformis linea, Amphora coffeiformis punctata, Amphora coffeiformis taylori, Amphora coffeiformis tenuis, Amphora delicatissima, Amphora delicatissima capitata, Amphora sp., Anabaena, Ankistrodesmus, Ankistrodesmus falcatus, Boekelovia hooglandii, Borodinella sp., Botryococcus braunii, Botryococcus sudeticus, Carteria, Chaetoceros gracilis, Chaetoceros muelleri, Chaetoceros muelleri subsalsum, Chaetoceros sp., Chlorella anitrata, Chlorella Antarctica, Chlorella aureoviridis, Chlorella candida, Chlorella capsulate, Chlorella desiccate, Chlorella ellipsoidea, Chlorella emersonii, Chlorella fusca, Chlorella fusca var. vacuolata, Chlorella glucotropha, Chlorella infusionum, Chlorella infusionum var. actophila, Chlorella infusionum var. auxenophila, Chlorella kessleri, Chlorella lobophora, Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella parva, Chlorella photophila, Chlorella pringsheimii, Chlorella Protothecoides, Chlorella protothecoides var. acidicola, Chlorella regularis, Chlorella regularis var. minima, Chlorella regularis var. umbricata, Chlorella reisiglii, Chlorella saccharophila, Chlorella saccharophila var. ellipsoidea, Chlorella sauna, Chlorella simplex, Chlorella sorokiniana, Chlorella sp., Chlorella sphaerica, Chlorella stigmatophora, Chlorella vanniellii, Chlorella vulgaris, Chlorella vulgaris, Chlorella vulgaris f. tertia, Chlorella vulgaris var. autotrophica, Chlorella vulgaris var. viridis, Chlorella vulgaris var. vulgaris, Chlorella vulgaris var. vulgaris f. tertia, Chlorella vulgaris var. vulgaris f. viridis, Chlorella xanthella, Chlorella zofingiensis, Chlorella trebouxioides, Chlorella vulgaris, Chlorococcum infusionum, Chlorococcum sp., Chlorogonium, Chroomonas sp., Chrysosphaera sp., Cricosphaera sp., Cryptomonas sp., Cyclotella cryptica, Cyclotella meneghiniana, Cyclotella sp., Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Dunaliella tertiolecta, Eremosphaera viridis, Eremosphaera sp., Ellipsoidon sp., Euglena, Franceia sp., Fragilaria crotonensis, Fragilaria sp., Gleocapsa sp., Gloeothamnion sp., Hymenomonas sp., Isochrysis aff. galbana, Isochrysis galbana, Lepocinclis, Micractinium, Micractinium, Monoraphidium minutum, Monoraphidium sp., Nannochloris sp., Nannochloropsis salina, Nannochloropsis sp., Navicula acceptata, Navicula biskanterae, Navicula pseudotenelloides, Navicula pelliculosa, Navicula saprophila, Navicula sp., Nephrochloris sp., Nephroselmis sp., Nitschia communis, Nitzschia alexandrina, Nitzschia communis, Nitzschia dissipata, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp., Ochromonas sp., Oocystis parva, Oocystis pusilla, Oocystis sp., Oscillatoria limnetica, Oscillatoria sp., Oscillatoria subbrevis, Pascheria acidophila, Pavlova sp., Phagus, Phormidium, Platymonas sp., Pleurochrysis carterae, Pleurochrysis dentate, Pleurochrysis sp., Prototheca wickerhamii, Prototheca stagnora, Prototheca portoricensis, Prototheca moriformis, Prototheca zopfii, Pyramimonas sp., Pyrobotrys, Sarcinoid chrysophyte, Scenedesmus armatus, Spirogyra, Spirulina platensis, Stichococcus sp., Synechococcus sp., Tetraedron, Tetraselmis sp., Tetraselmis suecica, Thalassiosira weissflogii, and Viridiella fridericiana.

One or more embodiments of the present invention involve a method comprising one or more of the following steps: providing an algae, providing a medium, providing a mineral medium, growing an algae within a medium, providing an FFA-containing feedstock to an algae-containing medium, allowing the algae to reduce the FFA content of the FFA-containing feedstock, and producing an algal product.

One or more of the following steps can also be utilized with the present invention: autoclaving a mineral medium, filtering particles from a FFA-containing feedstock, aerating an algae culture, agitating an algae-containing medium, providing a surfactant to a medium, and controlling the pH of the algae-containing medium.

In one or more embodiments, the medium can be autoclaved at 121° C. for 15 min. In one or more embodiments, filter-sterilized air, at a flow rate of from 0.05 or more to 2 or less based on the volume of gas per volume of liquid per minute (VVM), can be bubbled into the culture for aeration. In one or more embodiments, mixing can be provided by a magnetic stir bar at 250 rpm. In one or more embodiments, the pH of the medium can be controlled at 5.5, such as by automatic 0.05 N acid/base additions. In one or more embodiments, a nonionic surfactant, such as Tween® 80, known by the chemical names of polysorbate 80, polyoxyethylene (20) sorbitan monooleate, or (x)-sorbitan mono-9-octadecenoate poly(oxy-1,2-ethanediyl)), can be added to the medium to enhance the dispersion of oil into smaller droplets, such that the oil will become easier to ingest by the alga.

A wide range of useful algae products can be developed from the algae and the treated feedstock. Examples of algal products include algal biomass, dry algal cells, algal proteins, algal lipids, and algal carbohydrates. Algal biomass and algal lipid can be further converted into biofuel. Other examples of algal products include specialty substances with nutritional, pharmaceutical, cosmetic, and industrial uses.

Biomass can be generally described as biological material derived from living, or recently living organisms. With respect to algal biomass, it can be described as the wet algal cell mass separated from the aqueous medium in which the algal cells are cultivated or as the totally or partially dried algal cell mass. Biomass can be used as an energy source directly via combustion or co-combustion with other fuel to produce heat, or indirectly after converting the biomass to biofuel.

Biofuel can be generally described as fuel that contains energy from geologically recent carbon fixation. Biomass can be converted to biofuel and other energy containing substances in three different ways: thermal conversion, chemical conversion, and biochemical conversion.

Lipids may be broadly defined as hydrophobic or amphiphilic small molecules. The main biological functions of lipids include storing energy, signaling, and acting as structural components of cell membranes. Lipids can be particularly converted to biofuel by extracting the lipids and reacting them with alcohols, such as methanol or ethanol, through well-known chemical or enzymatic processes to make biodiesel, i.e., methyl or ethyl esters of fatty acids.

The conditions, such as time, pH, temperature, and dissolved oxygen, of the algae and a combined mixture of the algae and an FFA-containing feedstock can be adjusted to any conditions that will physiologically support the algae.

The algae and the FFA-containing feedstock are combined for a period of time that can be selected based on the particular algae and high FFA feedstock that are utilized. In one or more embodiments, this timeframe is from 2 hours or more to 10 days or less. In one or more embodiments, this timeframe is from 6 hours or more to 3 days or less. In one or more embodiments, this timeframe is from 2 hours or more to 6 hours or less.

The pH of the algae and combined mixture should be maintained within the physiologically acceptable range for the algae. The pH can also affect the transport/uptake of the FFA-containing feedstock by algae.

The pH can be controlled by any means known in the art. In one or more embodiments, the pH of the algae and combined mixture is from 2.5 or more to 8.5 or less. In one or more embodiments, the pH of the algae and combined mixture is from 4.0 or more to 6.0 or less. In one or more embodiments, the pH of the algae and combined mixture is controlled at 5.5, or approximate thereto. In embodiments where algae is used to treat a residual FFA-containing aqueous phase that is generated, the pH of the aqueous phase needs to be adjusted to the physiologically acceptable range of the algae, before contacting the aqueous phase with the algae.

The temperature of the algae and combined mixture should be maintained within the physiologically acceptable range for the algae. The temperature can be controlled by any heating or cooling equipment as known in the art. Such equipment may employ temperature sensors, thermometers, thermocouples and the like to monitor temperature, further including heating and/or cooling elements to control the temperature of the medium as monitored by those elements. Cooling is normally achieved by running cold water or other fluids through tubes or plates that are in contact with the algae and combined mixture. Heating is often achieved either by running hot water or other fluids through tubes or plates that are in contact with the algae and combined mixture, or by using electrically heated tubes, plates or other surfaces.

In one or more embodiments, the algae and combined mixture are maintained at from 10° C. or more to 40° C. or less, in other embodiments, from 15° C. or more to 35 ° C. or less, and in still other embodiments, from 20° C. or more to 30° C. or less. In one or more embodiments, the algae and combined mixture are at a temperature of 20° C. or more. In one or more embodiments, the algae and combined mixture are at a temperature of 28° C. or less. It should be noted that different algae have different physiologically suitable and tolerable temperatures. The optimal temperatures may be adjusted if more thermophilic or more psychrophilic algae are used.

The dissolved oxygen content of the algae and combined mixture should be maintained within the physiologically acceptable range for the algae. The dissolved oxygen content can be controlled by any means known in the art. In one or more embodiments, the dissolved oxygen content of the algae and combined mixture is controlled by adjusting the aeration flow rate and/or oxygen partial pressure of the gas (air, pure oxygen or mixtures of air and oxygen) and/or by adjusting the speed of mechanical agitation. The aeration rate and agitation speed are maintained within the range that provides adequate mixing without damaging or killing the algae cells (due to high shear stress or other damaging mechanisms).

In one or more embodiments, the algae and combined mixture have a dissolved oxygen content of from 0.02 milligram per liter (mg/L) or more to 10 mg/L or less, in other embodiments, from 0.05 mg/L or more to 5 mg/L or less, and in still other embodiments, from 0.1 mg/L or more to 2 mg/L or less. In one or more embodiments, the algae and combined mixture have a dissolved oxygen content of 0.2 mg/L or more. In one or more embodiments, the algae and combined mixture have a dissolved oxygen content of 1.5 mg/L or less.

In one or more embodiments, an FFA-containing feedstock is treated with a coagulant such that the coagulant and the FFA form an amount of coagulant complex. This amount of coagulant complex, or most of the coagulant complex, is separated from a final oil phase. Upon forming, the coagulant complex is located in a middle phase between the oil and water that exist within the composition. The upper oil phase, without the middle phase, is then collected and used as the treated feedstock for the formation of a product, such as biodiesel.

Preferred coagulants include multivalent cations, particularly the salts of multivalent cations. These cations exist in their dissociated form, such that the cations can form a complex with the FFA. The formed complexes are more surface active and tend to form a middle phase in the gravity or centrifugation separated mixture as described herein. The coagulant also causes the oil to form smaller droplets, which is beneficial for potential later processing by algae.

In one or more embodiments, a coagulant includes a multivalent cation selected from the group consisting of Calcium, Iron, Magnesium, Aluminum, Cobalt, and Zinc. In one or more embodiments, a coagulant is a salt of a multivalent cation selected from the group consisting of calcium chloride, ferric chloride, aluminum sulfate, aluminum chloride, sodium aluminate, ferric sulfate, ferrous sulfate, ferric chloride sulfate, magnesium carbonate, alum, clay, and hydrated lime.

Another group of coagulants that can be used are natural and synthetic polymers with multiple cationic functional groups. Examples include polyacrylamides, polyamines (e.g., epichlorhydrin-dimethylamine (epi-DMA)), Poly-diallyl-dimethyl-ammonium chloride (polyDADMAC or polyDDA), polyethylenimines, chitin, and chitosan.

In order to perform an effective coagulant complexation, an FFA-containing feedstock should be neutralized or the pH should be raised to near or above the logarithmic acid dissociation constant (pKa) values of the FFA's. If an FFA-containing feedstock is not already neutralized, the feedstock can be neutralized with the addition of a base. In one or more embodiments, a FFA-containing feedstock can be neutralized by adding the appropriate amount of 0.1 N NaOH to the feedstock. The neutralization includes verifying that the feedstock is at a pH of 7 or approximate thereto, or bringing the pH to 7 or approximate thereto.

The temperature of the mixture of the coagulant and FFA-containing feedstock must be sufficient to create the necessary phase separation. In certain embodiments, mixtures at room temperature do no give sufficient phase separation. For other certain embodiments, the formation of phase separation between the oil phase and aqueous phase occurred at 50° C. In one or more embodiments, the temperature of the coagulant and FFA-containing feedstock is 50° C. or more; in other embodiments, 70° C. or more; and in other embodiments, 80° C. or more. In general, a higher temperature gives a larger acid value reduction for the treated feedstock.

The time that the mixture of the coagulant and FFA-containing feedstock is exposed to heat can also be adjusted based on desired results. In general, a longer heating time results in a larger acid value reduction for the treated feedstock, although this benefit may level off after six hours. The mixture should be heating for a sufficient time period as to form the phase separation and achieve the desired acid value reduction.

With respect to mixing time, it is preferred that the mixing time is sufficient to form the coagulation complex in the middle phase. However, a relatively longer mixing time results in more difficult phase separation, such as separating the treated oil from the middle phase. Again, the mixture should be mixed for a sufficient time period as to form the phase separation and achieve the desired acid value reduction. Increasing the mixing time generally results in a lower acid value for the treated feedstock, but this benefit may level off after three hours.

The coagulant should be present at a sufficient molar ratio as to form the complexes of the coagulant and FFA. In one or more embodiments, the molar ratio of coagulant to FFA is 1:3 or approximate thereto; in other embodiments, 1:2 or approximate thereto; in other embodiments, 1:1 or approximate thereto; and in other embodiments, 3:2 or approximate thereto. In one or more embodiments, the molar ratio of coagulant to FFA is 1:3 or more and in other embodiments 3:2 or less. In one or more embodiments, the molar ratio of coagulant to FFA is from 1:3 or more to 3:2 or less. It is believed that increasing the molar ratio beyond certain stoichiometric ratio ranges required for complexation does not affect the acid value reduction of the treated feedstock.

After the coagulant and FFA-containing feedstock achieve sufficient phase separation and the complexes of coagulant and FFA form in the middle phase, the upper oil phase is removed as the treated feedstock for the formation of a product. One such product is biodiesel. The oil phase feedstock is turned into biodiesel by known methods catalyzed by one or more of the following, in parallel or in series: base, acid, solid catalysts, enzymes, microbial cells, and in free or immobilized forms. The middle phase containing the complexes, the phase now comprising concentrated fatty acids, can then be further processed or used based on the coagulant that is utilized.

One or more embodiments of the present invention involve a method comprising one or more of the following steps: providing an FFA-containing feedstock, ensuring that the FFA-containing feedstock is at a pH of greater than the FFA's logarithmic acid dissociation constant (pKa), neutralizing the FFA-containing feedstock to a pH of greater than the FFA's pKa, combining a coagulant with the FFA-containing feedstock, forming a complex with the coagulant and FFA, forming phase separations including an oil phase, a middle phase, and an aqueous phase where the middle phase includes the complex of coagulant and FFA, separating the oil phase from the middle phase and aqueous phase, and using the treated oil phase as a feedstock to form a product.

In one or more embodiments, the FFA's pKa is from 4.5 or more to 5 or less. In one or more embodiments, the FFA's pKa is 5 or approximate thereto. In one or more embodiments, the FFA's pKa corresponds to a pH value of from 4.5 or more to 5 or less.

In one or more embodiments, the FFA's pKa corresponds to a pH value of 5 or approximate thereto. In one or more embodiments, the pH is at or brought to 6 or more. A pH of 6 or more can ensure that a significant majority of the FFA's are in their anionic forms for complexation. In one or more embodiments, the pH is at or brought to from 5 or more to 10 or less. In one or more embodiments, the pH is 10 or less as to not cause oil hydrolysis during a process. In one or more embodiments, the pH is 9 or less.

As discussed above and herein, carbon source can be used interchangeably with FFA-containing feedstock. Preferred carbon sources include oils and greases having a high FFA content, where a high FFA content is characterized as those oils and greases having an FFA content of 1 wt. % or more. One preferential carbon source is waste cooking oil. Any oils or greases having an FFA content of 0.1 wt. % or more, also described herein as an FFA-containing feedstock, can be treated with an algae in accordance with embodiments of the present invention. Useful oils and greases can include waste cooking oil, yellow grease, brown grease, and some seed or plant oils with more than 1% FFA.

The untreated feedstock and treated feedstock can be characterized by their FFA content and acid value (AV). These feedstocks can also be characterized by the decrease in the FFA content and acid value following the treatment. The following Equation 1 and Equation 2 can be used to calculate the FFA content and acid value.

$$\begin{matrix}Acid Value = \frac{( A - B ) \times N \times 56.1}{W} & ( Eq . 1 ) \\ FFA ( \% ) = \frac{( A - B ) \times N \times {MW}_{FA}}{10 \times W} & ( Eq . 2 )\end{matrix}$$

One procedure for calculating the above equations is based on a method recommended by the American Oil Chemists' Society. One 1 g oil sample is added to 50 mL isopropyl alcohol in a flask. Five drops of a 1% phenolphthalein solution in isopropanol are added as a pH indicator. Titration is done with 0.1 N KOH until the pink color persists. The volume (mL) of KOH solution added is recorded. The same is done for the blank (control) without the oil sample.

AV (mg KOH/g oil) and the FFA content (%) of the oil sample are then estimated by the above equations. In the above equations (Equations 1 and 2), N is the normality of KOH solution used (e.g. 1); W is the oil sample weight (g) used (e.g. 1); A and B are the titration KOH volumes (mL) for the sample and the blank, respectively; and MW_{FA} is 282, MW of oleic acid, which is used to roughly represent the average MW of free fatty acids in the oil.

In one or more embodiments, the untreated composition has an FFA content of 1 wt % or more; in other embodiments, 5 wt % or more; and in other embodiments, 10 wt % or more. In one or more embodiments, the treatment results in the treated feedstock having an FFA content of 5 wt. % or less; in other embodiments, 4 wt. % or less; in other embodiments, 3 wt. % or less; in other embodiments, 1.5 wt. % or less; and in other embodiments, 1 wt. % or less.

In one or more embodiments, the untreated feedstock has an acid value of 20 mg KOH/g oil or more; in other embodiments, 10 mg KOH/g oil or more; and in other embodiments, 2 mg KOH/g oil or more. In one or more embodiments, the treatment results in the treated feedstock having an acid value of 8 mg KOH/g oil or less; in other embodiments, 6 mg KOH/g oil or less; in other embodiments, 4 mg KOH/g oil or less; in other embodiments, 2 mg KOH/g oil or less; and in other embodiments, 1 mg KOH/g oil or less.

In one or more embodiments, the treatment results in the treated feedstock having an acid value of 2.8 mg KOH/g oil, or approximate thereto, less than the acid value of the untreated feedstock. In one or more embodiments, the treatment results in the treated feedstock having an acid value of 2.4 mg KOH/g oil, or approximate thereto, less than the acid value of the untreated feedstock.

In addition to lowering the FFA content of a composition, the present invention provides one or more of the following advantages: reducing the overall cost of biodiesel production from WCO. In one or more embodiments, WCO with a lower FFA content can be half of the cost compared to vegetable oil. In one or more embodiments, biodiesel produced from WCO with a lower FFA content is cheaper than petroleum diesel fuel. Lowering the FFA content of a composition can also have applications with respect to manufacturing or processes relating to cosmetics and food.

### EXAMPLES

### Example 1

### Algae:

As one example for demonstrating the present invention, the microalga Ochromonas danica was grown in 500 mL Erlenmeyer flasks at room temperature. The mineral medium used contained 0.2 g/L nitrilotriacetic acid, 0.3 g/L KH2PO4, 0.4 g/L MgCO3, 0.05 g/L CaCO3, 0.24 g/L NH4Cl, and 0.1 g/L MgSO4.7H2O. Two different carbon sources were compared. The two carbon sources were two different waste cooking oils (WCO) and 40 g/L of each waste cooking oil was provided. The waste cooking oils had initial acid values of 10.7 mg KOH/g oil (about 5.4% FFA content) and 3.9 mg KOH/g oil (about 2.0% FFA content), respectively. Filter-sterilized air (0.2 L/min) was bubbled into the culture for aeration. Mixing was provided by a magnetic stir bar at 250 rpm. Experiments were performed at ambient temperature and light. Unless otherwise stated, pH was controlled at 5.5 in all systems by automatic 0.05 N acid/base additions. Cells grown for 48-70 hours (1.1-1.7 million cells/mL), at the late growth phase or early stationary phase, were used to inoculate the bioreactors.

The two WCO samples used in the experiments were collected from restaurants. Particles in the WCO were removed by filtration (Grade 5V and #4 Whatman filter) prior to use for algal cultivation and biodiesel production. Once the mixing used in the culture system was stopped, a predominant majority of the algal cells attached to the rising oil droplets and became concentrated in a layer at the oil-water interface. The floated oil layer was collected with a pipette and then centrifuged mildly (at 300 g, to avoid breaking the algal cells) for 15 min to better separate the oil from the attached or entrapped cells. 10 g of the oil collected after centrifugation were further filtered to determine the reduction of FFA level by measuring the acid values of the alga treated oils.

The oils were dried by heating the oils to 100° C. for at least 15 min with continuous stirring. This was to prevent problems such as emulsion formation and low conversion during the biodiesel production reactions. The acid value for both pretreatment processes presented here was measured according to the procedure provided by American Oil Chemical Society (AOCS Cd 3a-63, 1988).

The profiles of acid value reduction over time for the two samples of WCO with initial acid values of 10.7 mg KOH/g WCO (about 5.4% FFA content) and 3.9 mg KOH/g WCO (about 2.0% FFA content) were found. The acid values of the remaining oil decreased by 2.8 mg KOH/g oil and 2.4 mg KOH/g oil, respectively. 0. danica cells engulfed oil droplets, grew rapidly on both oils, and decreased the acid values of the remaining oil.

For comparison, the acid value reduction profiles were also determined for WCO samples that only contacted the cell-free medium. This showed that some FFA or acid value-contributing materials were dissolved or preferentially emulsified because of their higher surface-activities in the aqueous medium. Thus, the acid value reduction profiles showed a fast initial reduction of acid values in both cell-free and cell-containing systems. In the algal systems, the supernatant of samples taken along the experiments became less turbid with time, indicating the colloidal and/or emulsified materials were consumed or engulfed by cells. Also, the WCO that had been treated with algal cells achieved larger acid value reductions. This shows the alga's ability to pretreat WCO and other oil and lipid sources by reducing FFA content for easier subsequent biodiesel production.

### Example 2

### Coagulant:

In one example, a coagulant was used with base-neutralized FFA of oils. Here, the salts of multivalent cations, such as CaCl₂ and FeCl₃, were used as the coagulants.

Two WCO samples having 8.6 mg KOH/g oil (about 4.3% FFA content) and 15.4 mg KOH/g oil (about 7.7% FFA content) were used. The sample (10 mL) was added into two 30 mL glass tubes. An equal volume of aqueous CaCl₂ solution was added to one tube and an equal volume of FeCl₃ solution was added to the other. The salt-to-FFA molar ratio used was 1:2 for CaCl₂ and 1:3 for FeCl₃. The pH of the mixtures was adjusted to 7 by the addition of 0.1 N NaOH, to neutralize the FFA in the oil samples.

The mixtures were then mildly mixed for up to 3 hours by clamping the tube to a vertical (slightly inclined) disk rotating at 40 rpm. The tubes were then taken off the rotary disk and allowed to stand in a dry-bath heating block (Fisher Scientific, USA) for up to 7 hours at 70° C., until the oil and water phases were clearly separated. This procedure created a turbid middle phase between the water and oil phases. Only the upper oil phase (without the middle phase) was collected with pipettes in glass tubes, dried at 100° C. for 15 minutes, and subjected to acid value measurements to determine the acid value reduction. The weight of collected oil was also measured to determine the oil loss during the process.

Duplicate experiments were carried out, with both WCO samples having initial acid values of 15.4 mg KOH/g oil (˜7.7 wt % FFA) and 8.6 mg KOH/g oil (˜4.3 wt % FFA), at the following conditions: 70° C., heating duration of 4 or 6 h respectively for CaCl₂ or FeCl₃ system, mixing duration of 3 h, coagulant salt-to-FFA molar ratio of 1:2 or 1:3 for CaCl₂ or FeCl₃ system respectively, oil-to-aqueous phase volumetric ratio of 1:1.

With CaCl₂, for the two oil samples, the acid value was lowered from 8.6 to 3.2 (±0.3) mg KOH/g oil (i.e., from about 4.3 wt % FFA to about 1.6 wt % FFA) and from 15.4 to 4.4 (±0.2) mg KOH/g oil (i.e., from about 7.7 wt % FFA to about 2.2 wt % FFA). When FeCl₃ was used, the acid value reduction was from 8.6 to 4.8 (±0.1) mg KOH/g oil (i.e., from about 4.3 wt % FFA to about 2.4 wt % FFA) and from 15.4 to 6.5 (±0.3) mg KOH/g oil (i.e., from about 7.7 wt % FFA to about 3.25 wt % FFA).

In these experiments, a middle phase was created between the oil and aqueous phases in the mixture. The average oil loss by weight for the oil with an initial acid value of 15.4 mg KOH/g oil was 14%±3% or 18%±2%, respectively, when FeCl₃ or CaCl₂ was used. For the oil sample having an initial acid value of 8.6 mg KOH/g oil, the oil loss by weight was 10%±2% or 14%±1%, respectively, when FeCl₃ or CaCl₂ was used.

In light of the foregoing, it should be appreciated that the present invention significantly advances the art by providing an improved method and system of reducing the free fatty acid content of a feedstock. While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

What is claimed is:

1. A method for reducing the free fatty acid content of a feedstock comprising the steps of
combining algae with a medium to form an algae-containing medium,
combining a free-fatty-acid-containing feedstock with the algae-containing medium to form an algae treatment feedstock,
allowing the algae to reduce the free-fatty-acid content of the algae treatment feedstock, and, after said step of allowing,
producing a product from the algae treatment feedstock.

2. The method of claim 1, wherein the medium comprises water, the method further comprising the steps of
agitating the algae treatment feedstock to form an upper oil phase, a lower aqueous phase, and a middle phase and
collecting said upper oil phase.

3. The method of claim 2, wherein the collected upper oil phase has a lower free fatty acid content than the free-fatty-acid-containing feedstock.

4. The method of claim 2, wherein the collected upper oil phase has a lower acid value than the free-fatty-acid-containing feedstock.

5. The method of claim 1, wherein the medium comprises water and waste cooking oil.

6. The method of claim 1, wherein the medium comprises a mineral medium.

7. The method of claim 6, wherein the mineral medium comprises one or more of the following components: glucose, MgCO₃, NH₄Cl, KH₂PO₄, nitrilotriacetic acid, peptone, MgSO₄.7H₂O, yeast extract, CaCO₃, Na₂EDTA, FeCl₃.6H₂O, H₃BO₃, thiamine, MnCl₂.4H₂O, ZnSO₄.7H₂O, CoCl₂, 6H₂O, Na₂MoO₄.2H₂O and biotin.

8. The method of claim 7, wherein the mineral medium comprises 10 g/L glucose, 0.4 g/L MgCO₃, 0.3 g/L NH₄Cl, 0.3 g/L KH₂PO₄, 0.2 g/L nitrilotriacetic acid, 0.5 g/L peptone, 0.1 g/L MgSO₄.7H₂O, 0.5 g/L yeast extract, 0.05 g/L CaCO₃, 4.4 mg/L Na₂EDTA, 3.15 mg/L FeCl₃.6H₂O, 0.97 mg/L H₃BO₃, 0.25 mg/L thiamine, 0.18 mg/L MnCl₂.4H₂O, 0.02 mg/L ZnSO₄.7H₂O, 0.01 mg/L CoCl₂.6H₂O, 6 μg/L Na₂MoO₄.2H₂O and 2.5 μg/L biotin.

9. The method of claim 6, further comprising one or more of the steps consisting of
autoclaving the mineral medium,
filtering particles from the free-fatty-acid-containing feedstock, and
aerating the algae treatment feedstock.

10. The method of claim 1, further comprising the step of providing a surfactant to the medium.

11. The method of claim 1, wherein the produced product is biodiesel.

12. A method for reducing the free fatty acid content of a feedstock comprising the steps of
combining a coagulant with a free-fatty-acid-containing feedstock and
forming a coagulant complex including the coagulant and free fatty acid.

13. The method of claim 12, wherein the free-fatty-acid-containing feedstock has a pH of 5 or greater.

14. The method of claim 13, further comprising the step of adding a base to the free-fatty-acid-containing feedstock to bring the feedstock to a pH of from 5 to 10.

15. The method of claim 12, further comprising the steps of
forming phase separations including an upper oil phase, a middle phase, and a lower aqueous phase, where the middle phase includes the coagulant complex,
collecting the upper oil phase, and
using the upper oil phase to produce a product.

16. The method of claim 15, wherein the produced product is biodiesel.

17. A method for producing biodiesel comprising the steps of
providing a free-fatty-acid-containing feedstock,
treating the free-fatty-acid-containing feedstock to reduce the free fatty acid content thereof, where the step of treating includes combining at least one of an algae and a coagulant to the free-fatty-acid-containing feedstock,
forming phase separations in the treated feedstock, the phase separations including an upper oil phase, a middle phase, and a lower aqueous phase,
collecting the upper oil phase, and
producing a biodiesel product from the upper oil phase.

18. The method of claim 17, wherein said step of producing the biodiesel product includes extracting lipids and reacting the lipids with an alcohol using a chemical or enzymatic process.

19. The method of claim 18, wherein the alcohol is selected from the group consisting of methanol and ethanol.
